(19) United States
(12) Patent Application Publication
(10) Pub. No.: US 2015/0346210 A1
(43) Pub. Date: Dec. 3, 2015

(54) MULTIPLEX ASSAY FOR IMPROVED SCORING OF TUMOR TISSUES STAINED FOR PD-L1

(71) Applicant: Ventana Medical Systems, Inc., Tucson, AZ (US)

(72) Inventors: Hiro Nitta, Tucson, AZ (US); Bharathi Vennapusa, Tucson, AZ (US); Eslie Dennis, Tucson, AZ (US)

(21) Appl. No.: 14/725,288

(22) Filed: May 29, 2015

Related U.S. Application Data
(60) Provisional application No. 62/005,701, filed on May 30, 2014.

(51) Int. Cl. G01N 33/58; G01N 1/30

(52) U.S. Cl. CPC G01N 33/58; G01N 1/30; G01N 2333/70596

(57) ABSTRACT

Multiplex assays for improved scoring of tumor tissues stained with PD-L1 featuring PD-L1 staining in a first color plus staining of one or more differentiating markers, such as a marker specific for tumor cells and a marker specific for immune cells, are disclosed. The differentiation between the tumor cells and immune cells may improve the ease of scoring, the accuracy and speed of scoring, and the reproducibility of scoring of PD-L1 positive samples for therapy purposes.

### CROSS-REFERENCE TO RELATED APPLICATIONS

This disclosure claims the benefit of U.S. 62/005,701, filed May 30, 2014, the contents of which are hereby incorporated by reference in their entirety.

### BACKGROUND OF THE INVENTION

1. Field of the Invention

The present disclosure relates to materials and methods for histochemically detecting and scoring PD-L1 expression in tumor tissues.

2. Description of Related Art

Programmed death 1 (PD-1) is a member of the CD28 family of receptors, which includes CD28, CTLA-4, ICOS, PD-1, and BTLA. Two cell surface glycoprotein ligands for PD-1 have been identified, PD-L1 and PD-L2, and have been shown to downregulate T cell activation and cytokine secretion upon binding to PD-1 (Freeman et al., J Exp Med 192:1027-34 (2000); Latchman et al., Nat Immunol 2:261-8 (2001); Carter et al., Eur J Immunol 32:634-43 (2002); Ohigashi et al., Clin Cancer Res 11:2947-53 (2005)). Both PD-L1 (B7-H1) and PD-L2 (B7-DC) are B7 homologs that bind to PD-1, but do not bind to other CD28 family members.

The PD-L1-PD1 pathway is involved in the negative regulation of some immune responses and may play an important role in the regulation of peripheral tolerance. Interaction of PD-L1 with PD1 results in inhibition of TCR-mediated proliferation and cytokine production. PD-L1 has been suggested to play a role in tumor immunity by increasing apoptosis of antigen-specific T-cell clones (Dong et al. Nat Med 8:793-800 (2002)). Indeed, PD-L1 expression has been found in several murine and human cancers, including human lung, ovarian and colon carcinoma and various myelomas (Iwai et al. PNAS 99:12293-7 (2002); Ohigashi et al. Clin Cancer Res 11:2947-53 (2005)). Thus, measuring the amount of PD-L1 protein in biological samples may aid in the early detection of cancer pathologies and may help assess the efficacy and durability of investigational drugs that inhibit the binding of the PD-L1 protein.

However, the use of PD-L1 protein expression as an accurate predictor for cancer DM #69976 and/or the efficacy of anti-PD-1 and anti-PD-L1 directed therapies remain challenging. For example, many tumor samples show PD-L1 staining in both tumor cells and immune cells. Differentiation of these two cell types may be difficult for pathologists, especially when both are present in the same sample.

### SUMMARY OF INVENTION

The present invention features multiplex assays for improved scoring of tumor tissues stained with PD-L1. The assays feature PD-L1 staining in a first color plus staining of a differentiating marker specific for tumor cells or immune cells, in a second color, and optionally, a second differentiating marker specific for tumor cells or immune cells, in a third color. The assays of the present invention help to differentiate between the PD-L1 positive tumor cells and the PD-L1 positive immune cells. This may improve the ability of samples to be scored more quickly, accurately, and with a greater degree of reproducibility as compared to scoring samples stained with PD-L1 alone.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE DRAWINGS

The application file contains at least one image executed in color. Copies of this patent or patent application with color images will be provided by the Office upon request and payment of the necessary fee.

FIG. 1 shows staining of a NSCLC tumor sample (PD-L1 is shown in brown (a), cytokeratins from pan keratin antibody are shown in red (b), and CD4 of immune cells is shown in green/blue (c)). Counterstain is diluted hematoxylin.

### DESCRIPTION OF PREFERRED EMBODIMENTS

The present disclosure relates generally to histochemical or cytochemical methods of labeling tumor samples to facilitate scoring of PD-L1 expression in tumor cells, immune cells, or both. Briefly, cells are labeled with binding entities specific for PD-L1 and: (1) at least one tumor cell marker; (2) at least one immune cell marker; or (3) at least one tumor cell marker and at least one immune cell marker. The binding entities are then visualized in the tumor samples by generating at least three distinct detectable signals: a first detectable signal that correlates with the location of PD-L1 binding entity; a second detectable signal that correlates with the location of the tumor cell-specific binding entity; and a third detectable signal that correlates with the location of the immune cell marker. Each of the detectable signals are distinguishable from one another. Optionally, a counterstain may be provided in a fourth detectable signal and/or a fifth detectable signal may be generated from co-localization of any two of the first, second, and third detectable signals.

Tumor Samples

The present methods are compatible with tumor samples suitable for histochemical or cytochemical analysis, including, for example, fresh frozen, formalin-fixed paraffin-embedded (FFPE)), cytological smears (such as cervical smears), isolates of circulating tumor cells, etc. In a specific embodiment, the sample is a FFPE sample of tumor tissue.

Tumor Cell Markers and Immune Cell Markers

Any marker capable of distinguishing tumor cells from non-tumor cells may be used. Examples of tumor cell-specific biomarkers may include but are not limited to: cytokeratins detectable with the pan keratin antibody (e.g., basic cytokeratins, many of the acidic cytokeratins), other cytokeratins such as cytokeratin 7 (CK7) and cytokeratin 20 (CK20), chromogranin, synaptophysin, CD56, thyroid transcription factor-1 (TTF-1), p53, leukocyte common antigen (LCA), vimentin, smooth muscle actin, or the like (e.g., see Capelozzi, V., J Bras Pneumol. 2009; 35(4):375-382).

Any marker capable of distinguishing immune cells from non-immune cells may be used. Examples of immune cell-specific biomarkers may include but are not limited to: CD3, CD4, CD8, CD19, CD20, CD11c, CD123, CD56, CD14, CD33, or CD66b. In one example, a lymphocyte-specific marker is used. For example, a T-cell specific marker, such as CD3, CD4, or CD8, or a B-cell specific marker, such as CD19 or CD20 may be used.

In a specific embodiment, the immune cell marker is CD4 and the tumor cell marker is a cytokeratin detectable by a pan cytokeratin antibody.

Binding Entities

Histochemistry and cytochemistry are techniques often used to identify biomarkers within the context of intact cells by labeling the samples with molecules that bind specifically to the biomarker in a manner that can be visualized on a microscope. Immunohistochemistry (IHC) and immunocytochemistry (ICC) are types of histochemistry and cytochemistry that use antibodies to label the biomarkers. In situ hybridization (ISH) is a type of histochemistry or cytochemistry that uses nucleic acid probes to label specific nucleotide sequences in the tissue or cell sample. By identifying the biomarker in the context of a tissue environment or cellular environment, spatial relationships between the biomarkers and other morphological or molecular features of the cell or tissue sample can be elucidated, which may reveal information that is not apparent from other molecular or cellular techniques.

As used herein, the term “binding entity” shall refer to any compound or composition that is capable of specifically binding to a specific molecular structure in a tumor sample suitable for histochemical or cytochemical analysis. Examples include antibodies and antigen binding fragments thereof, as well as engineered specific binding structures, including ADNECTINs (scaffold based on 10^{th} FN3 fibronectin; Bristol-Myers-Squibb Co.), AFFIBODYs (scaffold based on Z domain of protein A from S. aureus; Affibody AB, Solna, Sweden), AVIMERs (scaffold based on domain A/LDL receptor; Amgen, Thousand Oaks, Calif.), dAbs (scaffold based on VH or VL antibody domain; GlaxoSmithKline PLC, Cambridge, UK), DAR Pins (scaffold based on Ankyrin repeat proteins; Molecular Partners AG, Zurich, CH), ANTICALINs (scaffold based on lipocalins; Pieris AG, Freising, DE), NANOBODYs (scaffold based on VHH (camelid Ig); Ablynx N/V, Ghent, BE), TI ANS-BODYs (scaffold based on Transferrin; Pfizer Inc., New York, N.Y.), SMIPs (Emergent Biosolutions, Inc., Rockville, Md.), and TETRANECTINs (scaffold based on C-type lectin domain (CTLD), tetranectin; Borean Pharma A/S, Aarhus, DK). Descriptions of such engineered specific binding structures are reviewed by Wurch et al, Development of Novel Protein Scaffolds as Alternatives to Whole Antibodies Imaging and Therapy: Status on Discovery Research and Clinical Validation, Current Pharmaceutical Biotechnology, Vol, 9, pp. 502-509 (2008).

In an embodiment, the binding entities are antibodies or antigen-binding fragments thereof. As used herein, the term “antibody” refers to any form of antibody that exhibits the desired biological or binding activity. Thus, it is used in the broadest sense and specifically covers, but is not limited to, monoclonal antibodies (including full length monoclonal antibodies), polyclonal antibodies, multispecific antibodies (e.g., bispecific antibodies), humanized antibodies, fully human antibodies, chimeric antibodies and camelized single domain antibodies.

As used herein, unless otherwise indicated, “antibody fragment” or “antigen binding fragment” refers to antigen binding fragments of antibodies, i.e. antibody fragments that retain the ability to bind specifically to the antigen bound by the full-length antibody, e.g. fragments that retain one or more CDR regions. Examples of antibody binding fragments include, but are not limited to, Fab, Fab′, F(ab′)2, and Fv fragments; diabodies; linear antibodies; single-chain antibody molecules, e.g., sc-Fv; nanobodies and multispecific antibodies formed from antibody fragments.

Exemplary anti-PD-L1 antibodies include SP263 (fully described in U.S. Provisional Patent Application Ser. No. 62/004,572, Docket Number 32151 US, and filed May 29, 2014, the disclosure of which is incorporated in its entirety herein by reference), SP142 (Cat. # M4420, Spring Biosciences, Inc., Pleasanton, Calif.), and PD-L1 (E1L3N®) XP® Rabbit mAb (Cat. # #13684; Cell Signaling Technologies, Inc., Danvers, Mass.). In a specific embodiment, the PD-L1 binding entity is SP263 or SP142, the tumor cell-specific binding entity is a pan keratin antibody, and the immune cell specific binding entity is an anti-CD4 antibody. In another specific embodiment, the PD-L1 binding entity is SP142, the tumor cell-specific binding entity is a pan keratin antibody, and the immune cell specific binding entity is an anti-CD4 antibody.

Visualization of Specific Binding Entities

As previously described, the assays of the present invention feature staining of PD-L1 as well as staining of one or more of tumor and immune cell markers to generate a detectable signal that correlates with the location at which an exogenous binding entity has bound to the sample. Histochemical and cytochemical methods of generating detectable signals from exogenous binding entities in samples are well known to one of ordinary skill in the art and typically involve application of one or more labels. Exemplary labels include chromogenic labels, fluorescent labels, luminescent labels, radiometric labels, etc., are used for recognition of the markers or targets (e.g., PD-L1, tumor cell-specific marker, immune cell-specific marker, etc.). Labels are well known to one of ordinary skill in the art and are not limited to the labels described herein. In a specific embodiment, the detectable signals are generated through use of chromogens.

In an embodiment, the label is applied through the use of a secondary antibody. For example, the binding entity may be a primary antibody specific for the PD-L1, immune cell marker, or tumor cell marker. If antibodies derived from different species of animal are used as the primary antibody, a secondary antibody specific for that species of antibody can be used to apply the label. In another example, the primary antibody can be modified to contain a separate moiety that can be bound by a specific binding entity. One example of such a primary antibody is a haptenized antibody (i.e., and antibody modified to contain a specific hapten). Many different haptens are known, so each of the primary antibodies can be modified to contain a different hapten, and different anti-hapten antibodies may be used to specifically label the different primary antibodies.

In another embodiment, the detectable signal may be amplified. As used herein, a signal is “amplified” when more label is deposited per primary antibody than by using a standard primary-secondary antibody arrangement. One commonly used method of amplification is tyramide signal amplification, which is described in Bobrow, M. N., Harris, T. D., Shaughnessy, K. J., and Litt, G. J. (1989) J. Immunol. Methods 125, 279-285. In an exemplary embodiment, a modified form of tyramide signal amplification as described in WO 2013148498 is used.

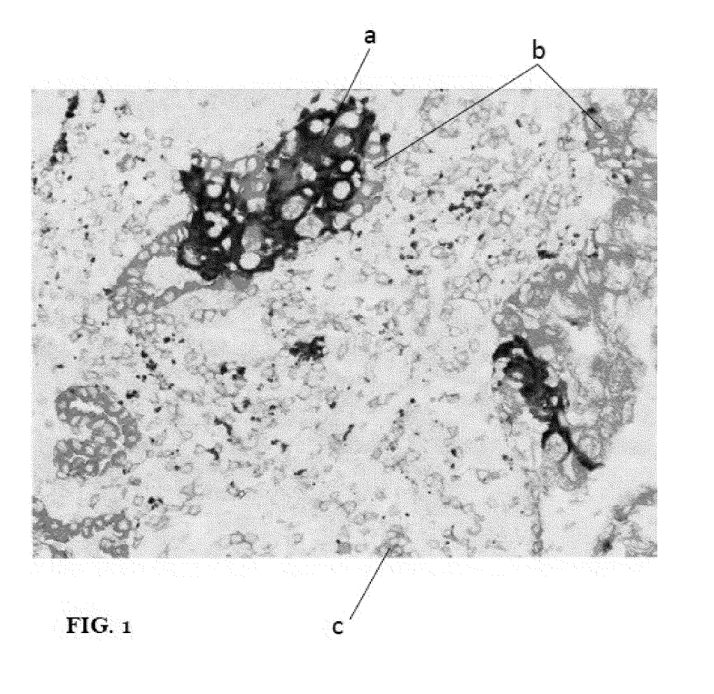

Referring now to FIG. 1, the present invention features multiplex assays for improved scoring of tumor tissues stained with PD-L1. The assays feature steps for staining PD-L1 in a first color (PD-L1 is shown in brown in FIG. 1), as well as steps for staining a tumor cell-specific marker and/or immune cell-specific marker with a second and/or third differentiating color. For example, FIG. 1 shows PD-L1 stained brown, cytokeratins targeted by pan keratin antibodies (cytokeratins specific for the epithelial cancer cells) stained in red/pink, and CD4 (of immune cells) stained in green/blue. The differentiating colors (red/pink and green/blue) allows one to determine if the PD-L1 that is detected is present in tumor cells or immune cells. Thus, the assays of the present invention help to differentiate between the PD-L1 positive tumor cells and the PD-L1 positive immune cells. This may improve the ability of samples to be scored (manual/visual, machine/image analysis) more quickly, accurately, and with a greater degree of reproducibility as compared to scoring samples stained with PD-L1 alone.

Table 1 illustrates the use of differentiating markers/colors for differentiating between the PD-L1 positive tumor cells and the PD-L1 positive immune cells. PD-L1 can be detected using the anti-PD-L1 antibody, and the PD-L1 is visible as a first color (e.g., brown in the example shown in FIG. 1). Tumor cells with PD-L1 will show the first color (PD-L1), as indicated by the “+” sign in Column 4 of Table 1 (note that the tumor cells without PD-L1 do not have the first color, as indicated by the “−” sign in Column 4 of Table 1). Immune cells also exhibit PD-L1 expression and can be shown as the first color (PD-L1), as indicated by the “+” sign in Column 1 of Table 1. To differentiate between the two cell types that are positive for PD-L1, the sample is stained for a tumor-specific marker (e.g., cytokeratins detected by the pan keratin antibody), which is visible as a second color (a color different from the first color). The sample may also be stained for an immune cell-specific marker (e.g., CD4 or other marker), which is visible as a third color (a color different from the first and second colors).

TABLE 1
(see summary below)

| | Column 1 | Column 2 | Column 3 | Column 4 |
|---|---|---|---|---|
| | Immune | Immune | Tumor | Tumor |
| | Cell with | Cell with- | Cell with | Cell With- |
| | PD-L1 | out PD-L1 | PD-L1 | out PD-L1 |
| PD-L1 (first color) | + | − | + | − |
| Tumor Cell-Specific | − | − | + | + |
| Differentiating Marker | | | | |
| (second color) | | | | |
| Immune Cell-Specific | + | + | − | − |
| Differentiating Marker | | | | |
| (third color) | | | | |

Table 1 Summary:

Cells that have both the first color (PD-L1) and the second color (tumor cell-specific differentiating marker) but not the third color (immune cell-specific differentiating marker) are PD-L1 positive tumor cells (Column 3); cells that have both the first color (PD-L1) and third color (immune cell-specific differentiating marker) but not the second color are PD-L1 positive immune cells (Column 1); and cells that have the second color (tumor cell-specific differentiating marker) but not the first color (PD-L1) nor the third color (immune cell-specific differentiating marker) are PD-L1 negative tumor cells (Column 4). Cells that have the third color but not the first color and second color are PD-L1 negative immune cells (Column 2). The present invention is not limited to staining in any particular order. For example, Example 1 describes staining first for PD-L1, then staining for the tumor cell-specific marker, then staining for the immune cell-specific marker, and finally using a counterstain. However, in some embodiments, the order of the staining is different. For example, in some embodiments, the immune cell-specific marker is stained before the tumor cell-specific marker is stained, etc.

As previously described, the assays of the present invention feature staining of PD-L1 as well as staining of one or more differentiating markers. Methods of staining may include immunohistochemisty (IHC), in situ hybridization (ISH), variations thereof, or any other appropriate staining or labeling technique. Such methods are well known to one of ordinary skill in the art. Staining techniques may be performed on various biological samples, such as tissue (e.g., fresh frozen, formalin-fixed paraffin-embedded (FFPE)) and cytological samples. Labels such as chromogenic labels, fluorescent labels, luminescent labels, radiometric labels, etc., are used for recognition of the markers or targets (e.g., PD-L1, tumor cell-specific marker, immune cell-specific marker, etc.). Labels are well known to one of ordinary skill in the art and are not limited to the labels described herein.

A non-limiting example of a detailed protocol is described in Example 1 below. Briefly, samples of interest are stained for PD-L1. The sample is incubated first with an anti-PD-L1 primary antibody. The anti-PD-L1 primary antibody is detected with a first color. In Example 1, the sample is incubated with a horseradish peroxidase (HRP)-conjugated secondary antibody against the primary anti-PD-L1 antibody and a substrate (3,3′-diaminobenzidine (DAB)) is added, producing the first color (e.g., brown). Alternative enzymes and substrates (and resulting colors are described below).

The sample is then stained for a first differentiating marker, e.g., a marker that is tumor cell-specific. Examples of tumor cell-specific biomarkers may include but are not limited to: cytokeratins detectable with the pan keratin antibody (e.g., basic cytokeratins, many of the acidic cytokeratins), other cytokeratins such as cytokeratin 7 (CK7) and cytokeratin 20 (CK20), chromogranin, synaptophysin, CD56, thyroid transcription factor-1 (TTF-1), p53, leukocyte common antigen (LCA), vimentin, smooth muscle actin, or the like (e.g., see Capelozzi, V., J Bras Pneumol. 2009; 35(4):375-382). The tumor cell-specific biomarkers are not limited to proteins detectable with IHC; for example, the tumor cell-specific biomarker may be a nucleic acid sequence of interest detectable with ISH techniques. Thus, methods describing IHC steps (e.g., incubating a sample with a primary antibody for a tumor cell-specific marker) may be substituted appropriately with ISH steps. One of ordinary skill in the art can substitute another appropriate tumor cell-specific biomarker for the cytokeratins as described in Example 1. The sample is incubated first with a primary antibody (e.g., anti-pan keratin antibody) against the first differentiating marker (tumor cell-specific marker). The anti-differentiating marker primary antibody is detected with a second color. In Example, 1, the sample is incubated with a haptenized antibody against the anti-differentiating marker primary antibody, and then the sample is incubated with an alkaline phosphatase (AP)-conjugated anti-hapten antibody. The substrate Fast Red Chromogen produces the second color (e.g., red).

The sample may then be stained for an immune cell-specific marker. Non-limiting examples of immune cell-specific biomarkers include CD4 or any other CD marker. Immune cell-specific biomarkers are well known to one of ordinary skill in the art. The immune cell-specific biomarkers are not limited to proteins detectable with IHC; for example, the immune cell-specific biomarker may be a nucleic acid sequence of interest detectable with ISH techniques. Thus, methods describing IHC steps (e.g., incubating a sample with a primary antibody for an immune cell-specific marker) may be substituted appropriately with ISH steps. One of ordinary skill in the art could substitute another appropriate immune cell-specific biomarker for CD4 as described in Example 1. The sample is incubated first with a primary antibody (e.g., anti-CD4 antibody) against the second differentiating marker (immune cell-specific marker). The anti-differentiating marker primary antibody is detected with a third color. In Example 1, the sample is incubated with a HRP-conjugated secondary antibody against the primary anti-differentiating marker antibody and the substrate HRP-Green Chromogen is added, producing the third color (e.g., green/blue).

In some embodiments, the samples are then counterstained, producing a fourth color (the fourth color being different from the first, second, and third colors). In some embodiments, the counterstain comprises hematoxyline; however, the counterstain is not limited to hematoxyline. Alternative counterstains are well known to one of ordinary skill in the art. For example, in some embodiments, the counterstain comprises methylene blue, nuclear red, toluidine blue, eosin, methyl green, or the like. The particular counterstain is generally selected to produce contrast so as to enhance visibility.

Following the staining procedure, the samples are then interpreted and scored (scoring is described below). In some embodiments, the results of the staining may be interpreted as described in Table 1. In the case of an assay that stains for PD-L1, a tumor cell-specific marker (e.g., cytokeratins), and an immune cell-specific marker, the cells that score for the first color and third color but not the second color are PD-L1 positive immune cells, cells that score for both the first color and the second color (but not the third color) are PD-L1 positive cancer cells, and cells that score for the second color but not the first color nor the third color are PD-L1 negative cancer cells. In some embodiments, the first color and second color overlap (or others overlap), producing a fifth (different) color. This overlap color may help scoring.

In some embodiments, the staining (e.g., PD-L1 and the differentiating marker) occurs sequentially. In some embodiments, the staining occurs simultaneously.

The present invention is not limited to staining in any particular order. For example, Example 1 describes staining first for PD-L1, then staining for the tumor cell-specific marker, then staining for the immune cell-specific marker, and finally using a counterstain. However, in some embodiments, the tumor cell-specific marker (or immune cell-specific marker) is stained first, followed by the PD-L1 staining, etc.

### Signaling Conjugates (IHC/ISH Chromogenic Substrates)

The present invention is not limited to the signaling conjugates (e.g., enzymes and chromogenic substrates) used in Example 1 nor to the other signaling conjugates described herein. Alternative enzyme-chromogenic substrate pairs for detection methods (e.g., immunohistochemistry, various in situ hybridization methods such as silver in situ hybridization (SISH), chromogenic in situ hybridization (CISH), fluorescence in situ hybridization (FISH), mRNA in situ hybridization, etc.) are well known to one of ordinary skill in the art.

Traditionally, chromogenic substrates precipitate when activated by the appropriate enzyme. That is, the traditional chromogenic substance is converted from a soluble reagent into an insoluble, colored precipitate upon contacting the enzyme. Chromogenic substrates used for the present invention may be compatible with automated slide staining instruments and processes and/or automated detection and analysis instruments and software. This may enable high detection sensitivity and multiplexing capability.

In some embodiments, the enzyme of the secondary antibody comprises HRP, alkaline phosphatase (AP), glucose oxidase, beta-galactosidase, the like, and/or others described in WO Patent Application No. 20131484498. In some embodiments, the substrate comprises DAB, Fast Red and Fast Blue, Fast Red and Black (silver), nitro blue tetrazolium chloride (NBT), 5-bromo-4-chloro-3-indolyl phosphate (BCIP), x-gal, 3-amino-9-ethylcarbazole (AEC), 5-bromo-4-chloro-3-indoyl-beta-D-galactopyranoside (BCIG), p-nitrophenyl phosphate (PNPP), 2,2′-azinobis[3-ethylbenzothiazoline-6-sulfonic acid] (ABTS), 3,3′,5,5′-tetramethylbenzidine (TMB), HRP-Green Chromogen, the like, and/or others described in WO Patent Application No. 20131484498. Example 2 (below) further describes alternative signaling conjugates.

The present invention is also not limited to any particular colors or color combinations. For example, in some embodiments, the first color (PD-L1) is brown; however, in some embodiments, the first color (PD-L1) may be any other appropriate color, e.g., red, blue, yellow, green, etc., depending on the enzyme-substrate combination. In some embodiments, the second color (e.g., cytokeratins, other markers) may be red/pink; however, in some embodiments, the second color (e.g., cytokeratins, other markers) may be any other appropriate color, e.g., brown, blue, yellow, green, etc., depending on the enzyme-substrate combination. In some embodiments, the third color (e.g., CD4, other markers) may be green/blue; however, in some embodiments, the third color (e.g., CD4, other markers) may be any other appropriate color, e.g., brown, red/pink, yellow, etc., depending on the enzyme-substrate combination. In some embodiments, the fourth color (counterstain) may be blue; however, the fourth color (counterstain) may be any other appropriate color, e.g., red, green, etc. In some embodiments, the fifth color (overlap of the two colors) may be purple (e.g., if the two colors are red and blue), green (e.g., if the two colors are yellow and blue), orange (e.g., if the two colors are red and yellow), etc., depending on the combination of the two colors.

### Scoring

Samples are then scored. The second color (and third color) directs scoring of PD-L1 in either the tumor cells or the immune cells. The use of the third color may improve scoring. For example, the use of the third color may help to clarify which cell type (tumor vs. immune) is PD-L1 positive. This can help the accuracy of the calculation of the number of PD-L1 positive immune cells, PD-L1 negative tumor cells, and PD-L1 positive tumor cells.

A positive result (e.g., a “PD-L1 positive result) may be calculated in a variety of ways and is not limited to the examples described herein.

In some embodiments, the number of PD-L1 positive tumor cells or the area of PD-L1 that is associated with tumor cells (e.g., the area of the slide covered with PD-L1 due to tumor cells) or the percentage of PD-L1 positive tumor cells may be calculated and then factored into an equation to calculate and H score. In some embodiments, if the H score is above a threshold for PD-L1 positivity then the sample is PD-L1 positive, and if the H score is below the threshold for PD-L1 positivity then the sample is PD-L1 negative.

Non-limiting examples of scoring calculations are presented in Example 3. Some examples are briefly described below:

In some embodiments, a positive result is determined by calculating the percentage of PD-L1 positive tumor cells and determining if that percentage is above the threshold for positivity or a predetermined cut-off. For example, in some embodiments, the minimum percentage of PD-L1 cells that confers PD-L1 positivity, e.g., 5% or more PD-L1 positive tumor cells confers PD-L1 positivity, 10% or more PD-L1 positive tumor cells confers PD-L1 positivity, 25% or more PD-L1 positive tumor cells confers PD-L1 positivity, 50% or more PD-L1 positive tumor cells confers PD-L1 positivity, etc.

In some embodiments, a positive result is determined by calculating the number of PD-L1 positive tumor cells divided by the total number of cells (e.g., number of tumor plus immune cells) and determining if that value is above the threshold for positivity (e.g., value greater than 0.15, value greater than 0.25, value greater than 0.5, etc.).

In some embodiments, a positive result is determined by calculating the sum of the percentage of PD-L1 positive tumor cells and PD-L1 positive immune cells and determining if that value is above the threshold for positivity (e.g., value greater than 40, value greater than 50, value greater than 60, etc.).

In some embodiments, a positive result is determined by calculating the number of PD-L1 positive tumor cells divided by the number of PD-L1 negative tumor cells and determining if that value is above the threshold for positivity (e.g., value greater than 1.5, value greater than 1.8, value greater than 2, etc.).

In some embodiments, a positive result is determined by calculating the number of PD-L1 positive tumor cells divided by the sum of the number of PD-L1 negative tumor cells and the number of PD-L1 negative immune cells and determining if that value is above the threshold for positivity (e.g., greater than 1.1, greater than 1.3, etc.).

In some embodiments, a positive result is determined by calculating the number of PD-L1 positive tumor cells divided by the sum of the number of PD-L1 negative tumor cells and the number of PD-L1 positive immune cells and determining if that value is above the threshold for positivity (e.g., greater than 1.1, greater than 1.3, etc.).

In some embodiments, a positive result is determined by calculating the number of PD-L1 positive tumor cells divided by the number of PD-L1 positive immune cells and determining if that value is above the threshold for positivity. In some embodiments, a positive result is determined by calculating the number of PD-L1 positive tumor cells divided by the number of PD-L1 negative immune cells and determining if that value is above the threshold for positivity.

As discussed above, PD-L1 positivity may be determined by calculating the percentage of PD-L1 positive tumor cells. In some embodiments, staining in greater than about 1% of cells (e.g., tumor cells) is scored as PD-L1 positive. In some embodiments, staining in greater than about 5% of cells (e.g., tumor cells) is scored as PD-L1 positive. In some embodiments, staining in greater than about 10% of cells (e.g., tumor cells) is scored as PD-L1 positive. In some embodiments, staining in greater than about 15% of cells (e.g., tumor cells) is scored as PD-L1 positive. In some embodiments, staining in greater than about 20% of cells (e.g., tumor cells) is scored as PD-L1 positive. In some embodiments, staining in greater than about 25% of cells (e.g., tumor cells) is scored as PD-L1 positive. In some embodiments, staining in greater than about 30% of cells (e.g., tumor cells) is scored as PD-L1 positive. In some embodiments, staining in greater than about 35% of cells (e.g., tumor cells) is scored as PD-L1 positive. In some embodiments, staining in greater than about 40% of cells (e.g., tumor cells) is scored as PD-L1 positive. In some embodiments, staining in greater than about 45% of cells (e.g., tumor cells) is scored as PD-L1 positive. In some embodiments, staining in greater than about 50% of cells (e.g., tumor cells) is scored as PD-L1 positive. In some embodiments, staining in greater than about 55% of cells (e.g., tumor cells) is scored as PD-L1 positive. In some embodiments, staining in greater than about 60% of cells (e.g., tumor cells) is scored as PD-L1 positive. In some embodiments, staining in greater than about 65% of cells (e.g., tumor cells) is scored as PD-L1 positive. In some embodiments, staining in greater than about 70% of cells (e.g., tumor cells) is scored as PD-L1 positive. In some embodiments, staining in greater than about 75% of cells (e.g., tumor cells) is scored as PD-L1 positive. In some embodiments, staining in greater than about 80% of cells (e.g., tumor cells) is scored as PD-L1 positive. In some embodiments, staining in greater than about 90% of cells (e.g., tumor cells) is scored as PD-L1 positive.

The number or percentage of PD-L1 positive immune cells may be relevant to the determination of PD-L1 positivity. In some embodiments, staining in greater than about 5% of immune cells is associated with scoring as PD-L1 positive. In some embodiments, staining in greater than about 10% of immune cells is associated with scoring as PD-L1 positive. In some embodiments, staining in greater than about 15% of immune cells is associated with scoring as PD-L1 positive. In some embodiments, staining in greater than about 25% of immune cells is associated with scoring as PD-L1 positive. In some embodiments, staining in greater than about 50% of immune cells is associated with scoring as PD-L1 positive.

The positivity of the sample may also be determined by the degree or intensity of staining (e.g., heavy staining may be positive and light staining may be negative). In some embodiments, scoring methods may feature scoring samples on an intensity scale, e.g., of 0 to 3, for PD-L1 expression (see for example, U.S. Provisional Patent Application No. 61/875,334 (Scoring Method For Mesothelin Protein Expression, the disclosure of which is incorporated in its entirety herein by reference). In some embodiments, samples are scored based on intensity and percentages of cells staining. For example, as described in U.S. Provisional Patent Application No. 61/875,334, H scores are calculated as: 1*(percentage of tumor cells staining at 1+ intensity)+2*(percentage of tumor cells staining at 2+ intensity)+3*(percentage of cells staining at 3+ intensity)=H score (a value between 0 and 300). Other scoring methods have been described and are well known to one of ordinary skill in the art.

### Computer-Based Immunodetection for Scoring

Imaging and detection and/or scoring may be done manually/visually or via a computer system. Examples of computer-based immunodetection for scoring are known to one of ordinary skill in the art. See, for example, U.S. Provisional Patent Application No. 62/005,222, Docket Number 32154 US (Automatic Field of View Selection Systems and Methods), the disclosure of which is incorporated in its entirety herein by reference, which describes detection of particular cells in a histopathology image with an automatic cell detection algorithm. For example, a sparse color unmixing algorithm is used to unmix the RGB image into different biological meaningful color channels. The automatic immune cell detection algorithm involves utilizing a cell detector that is trained using a convolutional neural network to identify the immune cells in the immune cell marker image channel. Further, the automatic immune cell detection algorithm involves utilizing a non-maximum suppression algorithm to obtain the immune cell coordinates from the probability map of immune cell presence generated from CNN classifier.

### Methods for Treating Patients

The scoring of patients for PD-L1 may be used to make therapeutic treatment decisions. One aspect of the present invention is that the scoring, described herein, is predictive of a therapeutic approach. In one embodiment, positive scoring is predictive of improved outcomes for PD-L1 inhibitor treatment therapies. A method, according to one embodiment, includes scoring a tumor sample for PD-L1 positivity and administering a therapy to patients having tumors that are scored positive for PD-L1.

The disclosed embodiments may further include identifying and/or selecting subjects for treatment with a PD-L1-targeted therapy (or a combination of PD-L1-targeted therapies), for example if the tumor sample obtained from the subject is scored using the methods provided herein. Additionally, the disclosed methods may further include administering one or more PD-L1-targeted therapies to the subject if the sample obtained from the subject is scored as being PD-L1 positive. In contrast, the disclosed embodiments may further include identifying subjects who will not likely benefit from treatment with a PD-L1-targeted therapy, for example if the tumor sample obtained from the subject is scored using the methods provided herein as PD-L1 negative.

PD-L1-targeted therapies include therapeutic agents that when administered in therapeutically effective amounts induce the desired response (e.g., treatment of a PD-L1-expressing tumor, for example by reducing the size or volume of the tumor, or reducing the size, volume or number of metastases).

In one example, a PD-L1-targeted therapy increases killing of PD-L1-expressing tumor cells (or reduces their viability). Such killing may need not result in 100% reduction of PD-L1-expressing tumor cells; for example PD-L1-targeted therapies that result in reduction in the number of viable PD-L1-expressing tumor cells by at least 10%, at least 20%, at least 30%, at least 40%, at least 50%, at least 75%, at least 90%, or at least 95% (for example as compared to no treatment with the PD-L1-targeted therapy) can be used in the methods provided herein. For example, the PD-L1-targeted therapy can reduce the growth of PD-L1-expressing tumor cells by at least 10%, at least 20%, at least 30%, at least 40%, at least 50%, at least 75%, at least 90%, or at least 95% (for example as compared to no treatment with the PD-L1-targeted therapy).

In one example, a PD-L1-targeted therapy decreases PD-L1expression or activity. Such inhibition need not result in 100% reduction of PD-L1 expression or activity; for example PD-L1-targeted therapies that result in reduction in PD-L1 expression or activity by at least 10%, at least 20%, at least 30%, at least 40%, at least 50%, at least 75%, at least 90%, or at least 95% (for example as compared to no treatment with the PD-L1-targeted therapy) can be used in the methods provided herein. For example, the PD-L1-targeted therapy can interfere with gene expression (transcription, processing, translation, post-translational modification), such as, by interfering with the PD-L1's mRNA and blocking translation of the gene product or by post-translational modification of a gene product, or by causing changes in intracellular localization.

Other examples of PD-L1-targeted therapies include inhibitory nucleic acid molecules, such as an antisense oligonucleotide, a siRNA, a microRNA (miRNA), a shRNA or a ribozyme. Such molecules can be used to decrease or eliminate PD-L1 gene expression. Any type of antisense compound that specifically targets and regulates expression of PD-L1 nucleic acid is contemplated for use. An antisense compound is one which specifically hybridizes with and modulates expression of a target nucleic acid molecule (such as PD-L1). These compounds can be introduced as single-stranded, double-stranded, circular, branched or hairpin compounds and can contain structural elements such as internal or terminal bulges or loops. Double-stranded antisense compounds can be two strands hybridized to form double-stranded compounds or a single strand with sufficient self-complementarity to allow for hybridization and formation of a fully or partially double-stranded compound. In some examples, an antisense PD-L1oligonucleotide is a single stranded antisense compound, such that when the antisense oligonucleotide hybridizes to a PD-L1mRNA, the duplex is recognized by RNaseH, resulting in cleavage of the mRNA. In other examples, a miRNA is a single-stranded RNA molecule of about 21-23 nucleotides that is at least partially complementary to an mRNA molecule that regulates gene expression through an RNAi pathway. In further examples, a shRNA is an RNA oligonucleotide that forms a tight hairpin, which is cleaved into siRNA. siRNA molecules are generally about 20-25 nucleotides in length and may have a two nucleotide overhang on the 3′ ends, or may be blunt ended. Generally, one strand of a siRNA is at least partially complementary to a target nucleic acid. Antisense compounds specifically targeting a PD-L1 gene can be prepared by designing compounds that are complementary to a PD-L1 nucleotide sequence, such as a mRNA sequence. PD-L1 antisense compounds need not be 100% complementary to the PD-L1 nucleic acid molecule to specifically hybridize and regulate expression of PD-L1. For example, the antisense compound, or antisense strand of the compound if a double-stranded compound, can be at least 75%, at least 80%, at least 85%, at least 90%, at least 95%, at least 99% or 100% complementary to a PD-L1nucleic acid sequence. Methods of screening antisense compounds for specificity are well known (see, for example, U.S. Publication No. 2003/0228689). In addition, methods of designing, preparing and using inhibitory nucleic acid molecules are within the abilities of one of skill in the art. Furthermore, sequences for PD-L1 are publicly available.

In some examples, the disclosed methods include providing a therapeutically effective amount of one or more PD-L1-targeted therapies to a subject having a PD-L1 positive result. Methods and therapeutic dosages of such agents and treatments are known to those of ordinary skill in the art, and for example, can be determined by a skilled clinician. In some examples, the disclosed methods further include providing surgery, radiation therapy, and/or chemotherapeutics to the subject in combination with the PD-L1-targeted therapy (for example, sequentially, substantially simultaneously, or simultaneously). Administration can be accomplished by single or multiple doses. Methods and therapeutic dosages of such agents and treatments are known to those skilled in the art, and can be determined by a skilled clinician. The dose required will vary from subject to subject depending on the species, age, weight and general condition of the subject, the particular therapeutic agent being used and its mode of administration.

Therapeutic agents, including PD-L1-targeted therapies, can be administered to a subject in need of treatment using any suitable means known in the art. Methods of administration include, but are not limited to, intradermal, transdermal, intramuscular, intraperitoneal, parenteral, intravenous, subcutaneous, vaginal, rectal, intranasal, inhalation, oral, or by gene gun. Intranasal administration refers to delivery of the compositions into the nose and nasal passages through one or both of the nares and can include delivery by a spraying mechanism or droplet mechanism, or through aerosolization of the therapeutic agent.

Administration of the therapeutic agents, including PD-L1-targeted therapies, by inhalant can be through the nose or mouth via delivery by spraying or droplet mechanisms. Delivery can be directly to any area of the respiratory system via intubation. Parenteral administration is generally achieved by injection. Injectables can be prepared in conventional forms, either as liquid solutions or suspensions, solid forms suitable for solution of suspension in liquid prior to injection, or as emulsions. Injection solutions and suspensions can be prepared from sterile powders, granules, and tablets. Administration can be systemic or local.

Therapeutic agents, including PD-L1-targeted therapies, can be administered in any suitable manner, for example with pharmaceutically acceptable carriers. Pharmaceutically acceptable carriers are determined in part by the particular composition being administered, as well as by the particular method used to administer the composition. Accordingly, there is a wide variety of suitable formulations of pharmaceutical compositions of the present disclosure. The pharmaceutically acceptable carriers (vehicles) useful in this disclosure are conventional. Remington's Pharmaceutical Sciences, by E. W. Martin, Mack Publishing Co., Easton, Pa., 15th Edition (1975), describes compositions and formulations suitable for pharmaceutical delivery of one or more therapeutic agents.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration can include ointments, lotions, creams, gels, drops, suppositories, sprays, liquids and powders. Conventional pharmaceutical carriers, aqueous, powder or oily bases, thickeners and the like may be necessary or desirable.

Therapeutic agents, including PD-L1-targeted therapies, for oral administration include powders or granules, suspensions or solutions in water or non-aqueous media, capsules, sachets, or tablets. Thickeners, flavorings, diluents, emulsifiers, dispersing aids or binders may be desirable.

Therapeutic agents, including PD-L1-targeted therapies, can be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

PD-L1-targeted therapies can be used in combination with additional cancer treatments (such as surgery, radiation therapy, and/or chemotherapy). In one example, the additional therapy includes one or more anti-tumor pharmaceutical treatments, which can include radiotherapeutic agents, anti-neoplastic chemotherapeutic agents, antibiotics, alkylating agents and antioxidants, kinase inhibitors, and other agents. Particular examples of additional therapeutic agents that can be used include alkylating agents, such as nitrogen mustards (for example, chlorambucil, chlormethine, cyclophosphamide, ifosfamide, and melphalan), nitrosoureas (for example, carmustine, fotemustine, lomustine, and streptozocin), platinum compounds (for example, carboplatin, cisplatin, oxaliplatin, and BBR3464), busulfan, dacarbazine, mechlorethamine, procarbazine, temozolomide, thiotepa, and uramustine; folic acid (for example, methotrexate, pemetrexed, and raltitrexed), purine (for example, cladribine, clofarabine, fludarabine, mercaptopurine, and tioguanine), pyrimidine (for example, capecitabine), cytarabine, fluorouracil, and gemcitabine; plant alkaloids, such as podophyllum (for example, etoposide, and teniposide); microtubule binding agents (such as paclitaxel, docetaxel, vinblastine, vindesine, vinorelbine (navelbine) vincristine, the epothilones, colchicine, dolastatin 15, nocodazole, podophyllotoxin, rhizoxin, and derivatives and analogs thereof), DNA intercalators or cross-linkers (such as cisplatin, carboplatin, oxaliplatin, mitomycins, such as mitomycin C, bleomycin, chlorambucil, cyclophosphamide, and derivatives and analogs thereof), DNA synthesis inhibitors (such as methotrexate, 5-fluoro-5′-deoxyuridine, 5-fluorouracil and analogs thereof); anthracycline family members (for example, daunorubicin, doxorubicin, epirubicin, idarubicin, mitoxantrone, and valrubicin); antimetabolites, such as cytotoxic/antitumor antibiotics, bleomycin, rifampicin, hydroxyurea, and mitomycin; topoisomerase inhibitors, such as topotecan and irinotecan; monoclonal antibodies, such as alemtuzumab, bevacizumab, cetuximab, gemtuzumab, rituximab, panitumumab, pertuzumab, and trastuzumab; photosensitizers, such as aminolevulinic acid, methyl aminolevulinate, porfimer sodium, and verteporfin, enzymes, enzyme inhibitors (such as camptothecin, etoposide, formestane, trichostatin and derivatives and analogs thereof), kinase inhibitors (such as imatinib, gefitinib, and erolitinib), gene regulators (such as raloxifene, 5-azacytidine, 5-aza-2′-deoxycytidine, tamoxifen, 4-hydroxytamoxifen, mifepristone and derivatives and analogs thereof); and other agents, such as alitretinoin, altretamine, amsacrine, anagrelide, arsenic trioxide, asparaginase, axitinib, bexarotene, bevacizumab, bortezomib, celecoxib, denileukin diftitox, estramustine, hydroxycarbamide, lapatinib, pazopanib, pentostatin, masoprocol, mitotane, pegaspargase, tamoxifen, sorafenib, sunitinib, vemurafinib, vandetanib, and tretinoin. Methods and therapeutic dosages of such agents are known to those skilled in the art, and can be determined by a skilled clinician. Other therapeutic agents, for example anti-tumor agents, that may or may not fall under one or more of the classifications above, also are suitable for administration in combination with the described specific binding agents. Selection and therapeutic dosages of such agents are known to those skilled in the art, and can be determined by a skilled clinician.

The assay results, findings, prognosis, predictions and/or treatment recommendations can be recorded and communicated to technicians, physicians and/or patients, for example. In certain embodiments, computers are used to communicate such information to interested parties, such as, patients and/or the attending physicians. Based on the prognosis of the PD-L1 tumor (such as whether the tumor is likely to respond to PD-L1-targeted therapy), the subject from whom the sample was obtained can be assigned a treatment plan, such as treatment or not with a PD-L1-targeted therapy.

In one embodiment, a prognosis, prediction and/or treatment recommendation based on the output value is communicated to interested parties as soon as possible after the assay is completed and the prognosis is generated. The results and/or related information may be communicated to the subject by the subject's treating physician. Alternatively, the results may be communicated directly to interested parties by any means of communication, including writing, such as by providing a written report, electronic forms of communication, such as email, or telephone. Communication may be facilitated by use of a suitably programmed computer, such as in case of email communications. In certain embodiments, the communication containing results of a prognostic test and/or conclusions drawn from and/or treatment recommendations based on the test, may be generated and delivered automatically to interested parties using a combination of computer hardware and software which will be familiar to artisans skilled in telecommunications. One example of a healthcare-oriented communications system is described in U.S. Pat. No. 6,283,761; however, the present disclosure is not limited to methods which utilize this particular communications system.

In certain embodiments of the methods of the disclosure, all or some of the method steps, including the assaying of samples, scoring of PD-L1 protein expression, prognosis of the tumor, and communicating of assay results or prognosis, may be carried out in diverse (e.g., foreign) jurisdictions.

### Kits for Scoring Pd-L1

The present invention also features a kit for scoring PD-L1. In some embodiments, the kit comprises an anti-PD-L1 antibody and one or two (or more) differentiating antibodies, e.g., an antibody directed to a tumor cell-specific marker, an antibody directed to an immune cell-specific marker, or both an antibody directed to a tumor cell-specific marker and an antibody directed to an immune cell-specific marker. In some embodiments, the kit further comprises secondary antibodies or other reagents for detection of the included primary antibodies. For example, the kit may comprise the secondary antibodies as well as the substrates used for detection (e.g., DAB, AEC, Fast Red, etc.). In some embodiments, the kit further comprises a counterstain. In some embodiments, the kit further comprises buffers appropriate for use with the included antibodies and/or other reagents.

In some embodiments, the kit further comprises amplifying reagents for amplifying the color (or other) signal of the enzyme-substrate reaction.

In some embodiments, the reagents of the kit are packaged in containers configured for use on an automated slide staining platform. For example, the containers may be dispensers configured for use on a BENCHMARK Series automated slide stainer.

In illustrative embodiments, the kit includes a series of reagents contained in different containers configured to work together to perform a particular assay. In one embodiment, the kit includes a labeling conjugate in a buffer solution in a first container. The buffer solution is configured to maintain stability and to maintain the specific binding capability of the labeling conjugate while the reagent is stored in a refrigerated environment and as placed on the instrument. In another embodiment, the kit includes a signaling conjugate in an aqueous solution in a second container. In another embodiment, the kit includes a hydrogen peroxide solution in a third container for concomitant use on the sample with the signaling conjugate. In the second or third container, various enhancers (e.g. pyrimidine) may be found for increasing the efficiency by which the enzyme activates the latent reactive species into the reactive species. In a further embodiment, the kit includes an amplifying conjugate.

### Example 1

### Protocol for Multiplex Assay

Example 1 describes a non-limiting example of a multiplex IHC assay of the present invention. A NSCLC sample slide is prepared according to standard protocols.

1. Apply 1 drop of PD-L1 SP142 antibody (Ventana Medical System, Tucson, Ariz.) to the slide and incubate for 16 minutes. Rinse slide with reaction buffer.

2. Apply 1 drop of OptiView HQ Universal Linker (Catalog No. 760-700, Ventana Medical Systems, Tucson, Ariz.) and incubate for 8 minutes. Rinse slide with Reaction Buffer.

3. Apply 1 drop of OptiView HRP Multimer (Catalog No. 760-700, Ventana Medical System, Tucson, Ariz.) and incubate for 8 minutes. Rinse slide with Reaction Buffer.

4. Apply 1 drop each of OptiView Amplifier H₂0₂ and OptiView Amplifier (Catalog No. 760-700, Ventana Medical System, Tucson, Ariz.) and incubate for 8 minutes. Rinse slide with Reaction Buffer.

5. Apply 1 drop of OptiView Amplifier Multimer (Catalog No. 760-700, Ventana Medical System, Tucson, Ariz.) and incubate for 8 minutes. Rinse slide with Reaction Buffer.

6. Apply 1 drop of OptiView H₂0₂ and 1 drop of OptiView DAB (Catalog No. 760-700, Ventana Medical Systems, Tucson, Ariz.) and incubate for 8 minutes. Rinse slide with Reaction Buffer.

7. Apply 1 drop of OptiView Copper (Catalog No. 760-700, Ventana Medical Systems, Tucson, Ariz.) and incubate for 4 minutes. Rinse slide with Reaction Buffer.

8. Apply 1 drop of Pan Keratin Antibody (AE1/AE3/PCK26) Primary Antibody (Catalog No. 760-2595, Ventana Medical Systems, Tucson, Ariz.). Incubate for 8 minutes. Rinse slide with Reaction Buffer.

9. Apply 1 drop of Haptenized anti-mouse antibody and incubate for 8 minutes. Rinse slide with reaction buffer.

10. Apply 1 drop of AP-conjugated anti-hapten antibody and incubate for 8 minutes. Rinse slide with Reaction Buffer.

11. Apply Fast Red chromogen and incubate for 8 minutes. Rinse with Reaction Buffer.

12. Apply 1 drop of anti-CD4 (SP35) rabbit monoclonal primary antibody (Catalog No. 790-4423, Ventana Medical Systems, Tucson, Ariz.) and incubate for 16 minutes. Rinse slide with Reaction Buffer.

13. Apply 1 drop of HRP-conjugated anti-rabbit antibody and incubate for 16 minutes. Rinse slide with Reaction Buffer.

14. Apply 2 drops of HRP-Green Chromogen Detection 1 and incubate for 4 minutes.

15. Apply 2 drops of HRP-Green Chromogen Detection 2 and incubate for 12 minutes. Rinse slide with Reaction Buffer.

16. Apply 1 drop of Mayer's Hematoxyline (1:5) and incubate for 4 minutes. Rinse slide with Reaction Buffer.

### Example 2

### Signaling Conjugates

The following example describes alternative signaling conjugates described in WO Patent Application No. 2013148498, the disclosure of which is incorporated in its entirety herein by reference.

In some embodiments, methods of detecting a target in a biological sample include contacting the biological sample with a detection probe, contacting the biological sample with a labeling conjugate, and contacting the biological sample with a signaling conjugate. The labeling conjugate includes an enzyme. The signaling conjugate includes a latent reactive moiety and a chromogenic moiety. The enzyme catalyzes conversion of the latent reactive moiety into a reactive moiety, which covalently binds to the biological sample proximally to or directly on the target. The method further includes illuminating the biological sample with light and detecting the target through absorbance of the light by the chromogenic moiety of the signaling conjugate. In one embodiment, the reactive moiety reacts with a tyrosine residue of the biological sample, the enzyme conjugate, the detection probe, or combinations thereof.

In some embodiments, the detection probe is an antibody probe. In some embodiments, the labeling conjugate includes an antibody coupled to the enzyme. Enzymes may include oxidoreductases, peroxidases, or hydrolases. An antibody for the labeling conjugate may be an anti-species or an anti-hapten antibody. The detection probe may include a hapten selected from the group consisting an oxazole hapten, pyrazole hapten, thiazole hapten, nitroaryl hapten, benzofuran hapten, triterpene hapten, urea hapten, thiourea hapten, rotenoid hapten, coumarin hapten, cyclolignan hapten, di-nitrophenyl hapten, biotin hapten, digoxigenin hapten, fluorescein hapten, and rhodamine hapten. In other examples, the detection probe is monoclonal antibody derived from a second species such as goat, rabbit, mouse, or the like. The labeling conjugate is configured, through its inclusion of an anti-species or an anti-hapten antibody to bind selectively to the detection probe.

Chromogen conjugates used for the present invention may be configured to absorb light more selectively than traditionally available chromogens. Detection is realized by absorbance of the light by the signaling conjugate; for example, absorbance of at least about 5% of incident light would facilitate detection of the target. In other darker stains, at least about 20% of incident light would be absorbed. Non-uniform absorbance of light within the visible spectra results in the chromophore moiety appearing colored. The signaling conjugates disclosed herein may appear colored due to their absorbance; the signaling conjugates may appear to provide any color when used in the assay, with certain particular colors including red, orange, yellow, green, indigo, or violet depending on the spectral absorbance associated with the chomophore moiety. According to another aspect, the chromophore moieties may have narrower spectral absorbances than those absorbances of traditionally used chromogens (e.g. DAB, Fast Red, Fast Blue). In illustrative embodiments, the spectral absorbance associated with the first chromophore moiety of the first signaling conjugate has a full-width half-max (FWHM) of between about 30 nm and about 250 nm, between about 30 nm and about 150 nm, between about 30 nm and about 100 nm, or between about 20 nm and about 60 nm.

Narrow spectral absorbances enable the signaling conjugate chromophore moiety to be analyzed differently than traditional chromogens. While having enhanced features compared to traditionally chromogens, detecting the signaling conjugates remains simple. In illustrative embodiments, detecting comprises using a bright-field microscope or an equivalent digital scanner. The narrow spectral absorbances enable chromogenic multiplexing at level beyond the capability of traditional chromogens. For example, traditional chromogens are somewhat routinely duplexed (e.g. Fast Red and Fast Blue, Fast Red and Black (silver), Fast Red and DAB). However, triplexed or three-color applications, or greater, are atypical, as it becomes difficult to discern one chromophore from another. In illustrative embodiments of the presently disclosed technology, the method includes detecting from two to at least about six different targets using different signaling conjugates or combinations thereof. In one embodiment, illuminating the biological sample with light comprises illuminating the biological sample with a spectrally narrow light source, the spectrally narrow light source having a spectral emission with a second full-width half-max (FWHM) of between about 30 nm and about 250 nm, between about 30 nm and about 150 nm, between about 30 nm and about 100 nm, or between about 20 nm and about 60 nm. In another embodiment, illuminating the biological sample with light includes illuminating the biological sample with an LED light source. In another embodiment, illuminating the biological sample with light includes illuminating the biological sample with a filtered light source.

In illustrative embodiments, detecting targets within the sample includes contacting the biological sample with a first amplifying conjugate that is covalently deposited proximally to or directly on the first labeling conjugate. The first amplifying conjugate may be followed by contacting the biological sample with a secondary labeling conjugate. Illustratively, the amplification of signal using amplifying conjugates enhances the deposition of signaling conjugate. The enhanced deposition of signaling conjugate enables easier visual identification of the chromogenic signal, that is, the amplification makes the color darker and easier to see. For low expressing targets, this amplification may result in the signal becoming sufficiently dark to be visible, whereas without amplification, the target would not be apparent. In one embodiment, the signaling conjugate is covalently deposited proximally to the target at a concentration of greater than about 1×10¹¹ molecules per cm²*μm to about 1×10¹⁶ molecules per cm²*μm of the biological sample. In one embodiment, the first target and the second target are genetic nucleic acids. Detecting the first target through absorbance of the light by the first signaling conjugate includes detecting a first colored signal selected from red, orange, yellow, green, indigo, or violet, the first colored signal associated with spectral absorbance associated with the first chromogenic moiety of the first signaling conjugate. Detecting the second target through absorbance of the light by the second signaling conjugate includes detecting a second colored signal selected from red, orange, yellow, green, indigo, or violet, the second colored signal associated with spectral absorbance associated with the second chromogenic moiety of the second signaling conjugate. Detecting an overlap in proximity through absorbance of the light by the first signaling conjugate overlapping in proximity with the second signaling conjugate so that a third colored signal associated with overlapping spectral absorbance of the first spectral absorbance and the second spectral absorbance. According to one example, this third color signals a normal genetic arrangement and the first and second colors signal a genetic rearrangement or translocation.

### Example 3

### Scoring

The following example describes various calculations (3A-3E) for determining PD-L1 positivity.

### Example 3A

Equation: PD-L1 Value=Percentage of PD-L1 positive tumor cells

Threshold for positivity: PD-L1 Value>40% is PD-L1 positive

A pathologist views Sample 3A and calculates the percentage of PD-L1 positive tumor cells as being 48%. Based on the threshold for positivity, Sample 3A is labeled PD-L1 positive.

### Example 3B

Equation: PD-L1 Value=# of PD-L1 positive tumor cells/total # of cells

Threshold for positivity: PD-L1 Value>0.25 is PD-L1 positive

A pathologist views Sample 3B. The number of PD-L1 positive tumor cells is 68, and the total number of cells is 460. The PD-L1 value based on the above calculation is 68/460=0.147. Based on the threshold for positivity, Sample 3B is labeled PD-L1 negative.

### Example 3C

Equation: PD-L1 Value=% of PD-L1 positive tumor cells+% PD-L1 positive immune cells

Threshold for positivity: PD-L1 Value>60 is PD-L1 positive

A pathologist views Sample 3C. The percent of PD-L1 positive tumor cells is 50, and the percent of PD-L1 positive immune cells is 20. The PD-L1 value based on the above calculation is 50+20=70. Based on the threshold for positivity, Sample 3C is labeled PD-L1 positive.

### Example 3D

Equation: PD-L1 Value=# of PD-L1 positive tumor cells/(# of PD-L1 negative tumor cells+# of PD-L1 positive immune cells)

Threshold for positivity: PD-L1 Value>0.8 is PD-L1 positive

A pathologist views Sample 3D. The number of PD-L1 positive tumor cells is 68, the number of PD-L1 negative tumor cells is 45, and the number of PD-L1 positive immune cells is 210. The PD-L1 value based on the above calculation is 68/(45+210)=0.266. Based on the threshold for positivity, Sample 3D is labeled PD-L1 negative.

### Example 3E

Equation: H score=1*(percentage of tumor cells staining at 1+ intensity)+2*(percentage of tumor cells staining at 2+ intensity)+3*(percentage of cells staining at 3+ intensity)

Threshold for positivity: H score>125 is PD-L1 positive

A pathologist views Sample 3E. The percentage of PD-L1 positive tumor cells staining at 1+ intensity is 5%, the percentage of PD-L1 positive tumor cells staining at 2+ intensity is 35%, and the percentage of PD-L1 positive tumor cells staining at 3+ intensity is 20%. The H score is 5(1)+2(35)+3 (20)=135. Based on the threshold for positivity, Sample 3E is labeled PD-L1 positive.

### REFERENCES

The disclosures of the following articles and patent documents are incorporated in their entirety by reference herein: (1) Capelozzi, V., Role of Immunohistochemistry in the diagnosis of lung cancer, J Bras Pneumol. 2009; 35(4): 375-382; (2) WO Patent Application No. 20131484498/U.S. Patent Application No. 2013/0260379 (Signaling Conjugates and Methods of Use); (3) U.S. Provisional Patent Application No. 62/005,222 Docket Number 32154 US (Automated Field of View Selection Systems and Methods); (4) U.S. Provisional Patent Application No. 61/875,334 Docket Number 31872 US (Scoring Method for Methothelin Protein Expression); Provisional Patent Application Ser. No. 62/004,572, Docket Number 32151 US, and filed May 29, 2014.

As used herein, the term “about” refers to plus or minus 10% of the referenced number. Each reference cited in the present application is incorporated herein by reference in its entirety.

Various modifications of the invention, in addition to those described herein, will be apparent to those skilled in the art from the foregoing description. Such modifications are also intended to fall within the scope of the appended claims. For example, an “antibody” used in accordance with the present invention may be a whole antibody or a fragment of an antibody that is effective in binding to a desired target site. Also, when appropriate, an “antibody” of the present invention may be substituted with a targeting moiety (e.g., ligand peptide, small molecule, etc.). For example, if the tumor cell or the immune cell has a specific, differentiating and unique cell surface receptor, then a corresponding targeting moiety may be used in accordance with the present invention to differentiate tumor cells from immune cells.

What is claimed is:

1. A multiplex method of labeling PD-L1 in a tumor tissue sample, said method comprising:
contacting the tissue sample with an anti-PD-L1 primary antibody; and
contacting the same tissue sample with
a primary antibody directed to a tumor cell-specific marker; or
a primary antibody directed to an immune cell-specific marker; or
a primary antibody directed to a tumor cell-specific marker and an antibody directed to an immune cell-specific marker; and
visualizing each of the antibodies in the tissue sample with a reagent that generates a detectable signal corresponding to each of the primary antibodies, wherein the anti-PD-L1 antibody has a first detectable signal, the antibody directed to the tumor cell-specific marker has a second detectable signal distinguishable from the first detectable signal, and the antibody directed to an immune cell-specific marker has a third detectable signal distinguishable from the first detectable signal and the second detectable signal.

2. The method of claim 1, wherein the tumor cell-specific marker is selected from the group consisting of a cytokeratin, chromogranin, synaptophysin, CD56, thyroid transcription factor-1 (TTF-1), p53, leukocyte common antigen (LCA), vimentin, and smooth muscle actin.

3. The method of claim 1, wherein the immune cell-specific marker is selected from the group consisting of CD3, CD4, CD8, CD19, CD20, CD11c, CD123, CD56, CD14, CD33, or CD66b.

4. The method of claim 1, wherein the immune cell-specific marker is a T-cell marker or a B-cell marker.

5. The method of claim 1, wherein the tissue sample is contacted with the antibody directed to the tumor cell-specific marker and the antibody directed to the immune cell-specific marker, wherein the antibody directed to the tumor cell-specific marker is a pan-keratin antibody and the antibody directed to the immune cell-specific marker is an anti-CD4 antibody.

6. The method of claim 1, wherein the anti-PD-L1 antibody is SP263 or SP142.

7. The method of claim 1, wherein the first, second, and third detectable signals are generated by chromogens.

8. The method of claim 7, wherein:
the first detectable signal is generated by contacting the tissue sample with a horseradish peroxidase (HRP)-conjugated secondary antibody and 3,3′-Diaminobenzidine (DAB), wherein the HRP catalyzes a reaction with DAB to produce a brown color
the second detectable signal is generated by:
contacting the sample with an alkaline phosphatase labeled antibody that recognizes the antibody directed against the labeled with alkaline phosphatase (AP);
reacting the alkaline phosphates with a Fast Red chromogen and naphthol to produce a red color; and
the third detectable signal is generated by contacting the sample with a HRP-conjugated secondary antibody and HRP-green chromogen, the HRP catalyzes a reaction with HRP-green chromogen to produce a green color.

9. The method of claim 1, wherein the first, second, and/or third detectable signal is an amplified signal.

10. The method of claim 9, wherein the amplified signal is generated by tyramide signal amplification.

11. The method of claim 1, wherein contacting the sample with the primary antibodies is performed simultaneously.

12. The method of claim 1, wherein contacting the sample with the primary antibodies is performed sequentially.

13. The method of claim 1 further comprising counterstaining the tissue sample, the counterstain producing a fourth detectable signal that is distinguishable from the first, second, and the third detectable signals.

14. The method of claim 13, wherein the counterstain comprises hematoxylin.

15. The method of claim 1, wherein a fifth detectable signal is produced by overlap of the first detectable signal and the second detectable signal.

16. The method of claim 1, wherein a sixth detectable signal is produced by overlap of the first detectable signal and the third detectable signal.

17. A method of scoring PD-L1 expression in a tumor sample, the method comprising labeling the tumor tissue sample according to claim 1 and scoring PD-L1 expression in tumor cells, immune cells, or both, wherein co-localization of the first and second detectable signals indicates the presence of PD-L1-positive tumor cells and co-localization of the first and third detectable signals indicates the presence of PD-L1-positive immune cells.

18. The method of claim 17, wherein the total number of PD-L1 positive and PD-L1-negative tumor cells is quantitated.

19. The method of claim 18, wherein the tumor is scored as PD-L1 positive if staining for PD-L1 is detected in greater than about 10% of tumor cells.

20. The method of claim 18, wherein the tumor is scored as PD-L1 positive if staining for PD-L1 is detected in greater than about 50% of tumor cells.

21. The method of claim 17, wherein the total number of PD-L1 positive and PD-L1-negative immune cells is quantitated.

22. The method of claim 21, wherein the tumor is scored as PD-L1 positive if staining for PD-L1 is detected in greater than about 10% of immune cells.

23. The method of claim 21, wherein the tumor is scored as PD-L1 positive if staining for PD-L1 is detected in greater than about 50% of immune cells.

24. The method of claim 17, wherein PD-L1-positive immune cells, PD-L1 positive tumor cells, and PD-L1 negative tumor cells are quantitated to generate a PD-L1 Value, wherein:
PD-L1 Value=PD-L1 positive tumor cells/(PD-L1 negative tumor cells+PD-L1 positive immune cells),
wherein:
PD-L1 positive tumor cells is calculated either by counting the number of cells staining for both the first and second detectable signals or by calculating the area of the tissue sample in which the first detectable signal is associated with the second detectable signal;
PD-L1 negative tumor cells is calculated either by counting the number of cells staining for the second detectable signal only or by calculating the area of the tissue sample in which the second detectable signal is not associated with the first detectable signal; and
PD-L1 positive immune cells is calculated either by counting the number of cells staining for both the first and third detectable signals or by calculating the area of the tissue sample in which the first detectable signal is associated with the third detectable signal.

25. The method of claim 17, further comprising scoring intensity of PD-L1 staining in PD-L1 positive tumor cells and calculating an H score, wherein:
H score=1*(percentage of PD-L1 positive tumor cells staining at 1+ intensity)+2*(percentage of PD-L1 positive tumor cells staining at 2+ intensity)+3*(percentage of PD-L1 positive tumor cells staining at 3+ intensity).
